(19) United States
(12) Patent Application Publication
(10) Pub. No.: US 2011/0015247 A1
(43) Pub. Date: Jan. 20, 2011

(54) NOVEL CRYSTALLINE FORM OF CARVEDILOL DIHYDROGEN PHOSPHATE AND RELATED PROCESSES

(75) Inventors: Giridhar Thota, Hyderabad (IN); Srinivasulu Gudipati, Hyderabad (IN); Srinivasa Rao Kotaru, Hyderabad (IN)

Correspondence Address: Kaplan Gilman & Pergament LLP, 1480 Route 9 North, Woodbridge, NJ, 07095, US

(73) Assignee: SHODHANA LABORATORIES LIMITED, Hyderabad (IN)

(21) Appl. No.: 12/935,171

(22) PCT Filed: Aug. 26, 2008
(86) PCT No.: PCT/IN2008/000539

§ 371 Date: Sep. 28, 2010

(30) Foreign Application Priority Data
Apr. 4, 2008 (IN) 861/CHE/2008

(51) Int. Cl. A61K 31/403; C07D 209/88; A61P 9/12

(52) U.S. Cl. 514/411; 548/444

(57) ABSTRACT

The present patent application relates to a novel crystalline form (Form S) of Carvedilol dihydrogen phosphate and a process for its preparation. It also relates to an improved process for the preparation of Carvedilol dihydrogen phosphate.

### FIELD OF THE INVENTION

The present patent application relates to a novel crystalline form of Carvedilol dihydrogen phosphate and a process for its preparation. It also relates to an improved process for the preparation of Carvedilol dihydrogen phosphate

### BACKGROUND OF THE INVENTION

Carvedilol dihydrogen phosphate is chemically described as 1-(carbazol-4-yloxy-3-[[2-(o-methoxyphenyloxy)ethyl]amino]-2-propanol dihydrogen phosphate and is represented by the following structural Formula I

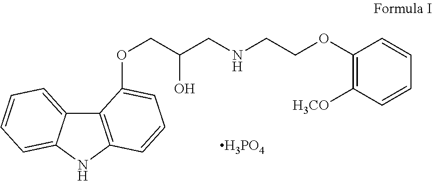

Carvedilol is an antihypertensive drug used in the treatment of cardiovascular diseases, including congestive heart failure, hypertension (high blood pressure) and angina. Carvedilol dihydrogen phosphate developed under the trade name [COREG-CR] developed by GSK, was superior to Carvedilol [COREG] in controlling the dissolution and adsorption of COREG in the body and also benefits in having less dosage plan for COREG-CR.

U.S. Pat. No. 4,503,067 discloses Carvedilol and process for its preparation that involves reaction of 4-(2,3-epoxy propoxy)carbazole with 2-(2-methoxy phenoxy ethylamine).

U.S. Pat. No. 7,268,156 B2 discloses the salts and solvates of Carvedilol including crystalline Carvedilol dihydrogen phosphate hemihydrate and process for its preparation that include reaction of Carvedilol with aqueous phosphoric acid in water.

Various crystalline forms and amorphous form of Carvedilol dihydrogen phosphate are described in patent applications including US2006/0182804 A1 WO2007/144900 A2; US2008/0167477 A1; WO2008/070072 A2 and WO2008/083130.

It is the endeavor of pharmaceutical scientists to provide a novel and stable crystalline form of drug substances, more specifically, thermodynamically stable forms of drug substances which would have thermodynamic stability and enhanced solubility.

Therefore, there remains a continuing need to provide polymorphs of Carvedilol dihydrogen phosphate and processes for their preparation, which are inexpensive, suitable for commercial manufacturing.

### SUMMARY OF THE INVENTION

In one aspect, the present patent application provides a novel crystalline form of Carvedilol dihydrogen phosphate (Form S) characterized by having an X-ray powder diffraction pattern comprising peak intensities expressed in degrees 2θ that are selected from 6.6±0.2, 8.1±0.2, 9.2±0.2, 13.0±0.2, 14.9±0.2, 19.7±0.2, 22.2±0.2 and 28.1±0.2.

The crystalline form S of Carvedilol dihydrogen phosphate is further characterized by a single melting endotherm peak between about 145° C. and about 153° C. as measured by differential scanning calorimetry.

The crystalline form S of Carvedilol dihydrogen phosphate characterized further by an FT-IR spectrum that comprises at least one absorption band selected from the group consisting of 521.7, 727.1, 751.3, 786.9, 1024.2, 1051.2, 1100.4, 1125.5, 1219.0, 1253.7, 1332.8, 1454.3, 1505.4, 1605.7 and 1628.9±0.2 cm⁻¹.

In second aspect the present application provides a process for the preparation of crystalline form of Carvedilol dihydrogen phosphate (Form S) comprising the steps of:

a) providing a solution of Carvedilol dihydrogen phosphate in a solvent comprising alcohol;

b) causing the solution to precipitate; and

c) recovering the precipitated solid form.

In third aspect there is provided a process for the preparation of Carvedilol dihydrogen phosphate comprises of:

a) reacting 4-(2,3-epoxy propoxy)carbazole with 2-(2-methoxy phenoxy)ethylamine to obtain a solution comprising Carvedilol;

b) reacting the Carvedilol solution obtained in step a) with phosphoric acid.

In fourth aspect the present application provides pharmaceutical composition comprising crystalline form of Carvedilol dihydrogen phosphate (Form S) and pharmaceutically acceptable carrier.

### BRIEF DESCRIPTION OF THE DRAWINGS

FIG. 1 is an illustrative X-ray powder diffraction pattern of Carvedilol dihydrogen phosphate crystalline Form S obtained in Example 2.

FIG. 2 is an illustrative DSC thermogram of Carvedilol dihydrogen phosphate crystalline Form S obtained in Example 2.

FIG. 3 is an illustrative Infrared spectrum of Carvedilol dihydrogen phosphate crystalline Form S obtained in Example 2.

FIG. 4 is an illustrative thermo gravimetric curve of Carvedilol dihydrogen phosphate crystalline Form S obtained in Example 2.

### DETAILED DESCRIPTION

As used herein the terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

All percentages and ratios used herein are by weight of the total composition and all measurements made are at 25° C. and normal pressure unless otherwise designated. All temperatures are in Degrees Celsius unless specified otherwise.

The crystallographic and powder X-ray diffraction arts, shifts in peak positions or the relative intensities of one or more peaks of a pattern can occur because of, without limitation: the equipment used, the sample preparation protocol, preferred packing and orientations, the radiation source, operator error, method and length of data collection, and the like. However, those of ordinary skill in the art should be able to compare the figures herein with a pattern generated of an unknown form. The same holds true for other techniques which may be reported herein.

In one aspect the present patent application provides a novel crystalline form of Carvedilol dihydrogen phosphate (Form S) characterized by having an X-ray powder diffraction pattern comprising peak intensities expressed in degrees 2θ that are selected from 6.6±0.2, 8.1±0.2, 9.2±0.2, 13.0±0.2, 14.9±0.2, 19.7±0.2, 22.2±0.2 and 28.1±0.2.

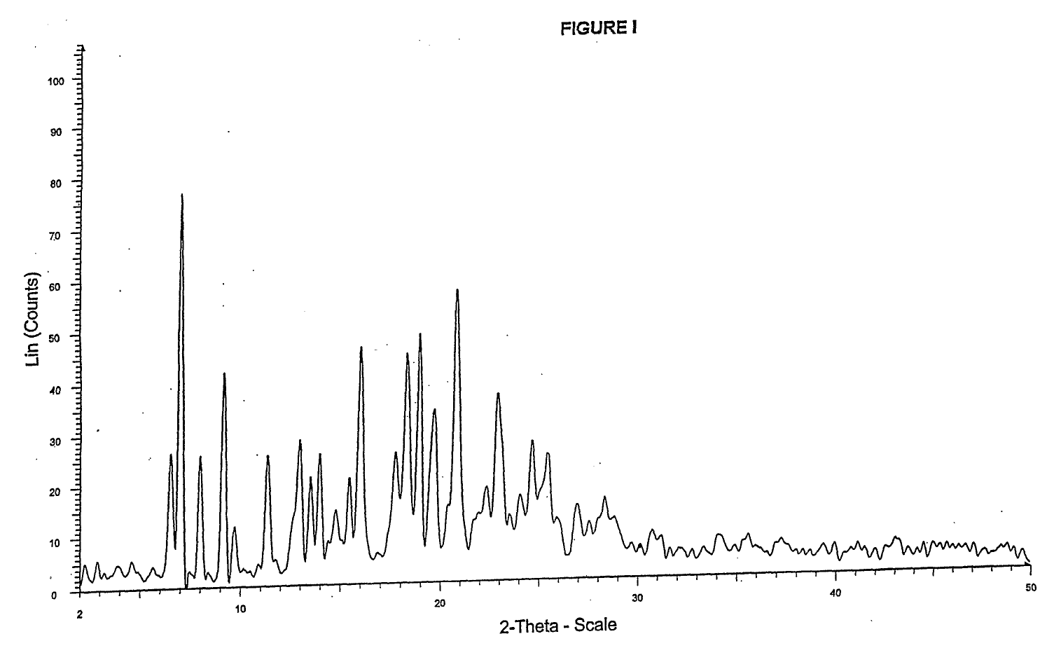

Crystalline Form S of Carvedilol dihydrogen phosphate of the present application is further characterized by XRPD pattern as shown substantially in FIG. 1.

The crystalline form S of Carvedilol dihydrogen phosphate is further characterized by a single melting endotherm peak between about 145° C. and about 153° C. as measured by differential scanning calorimetry.

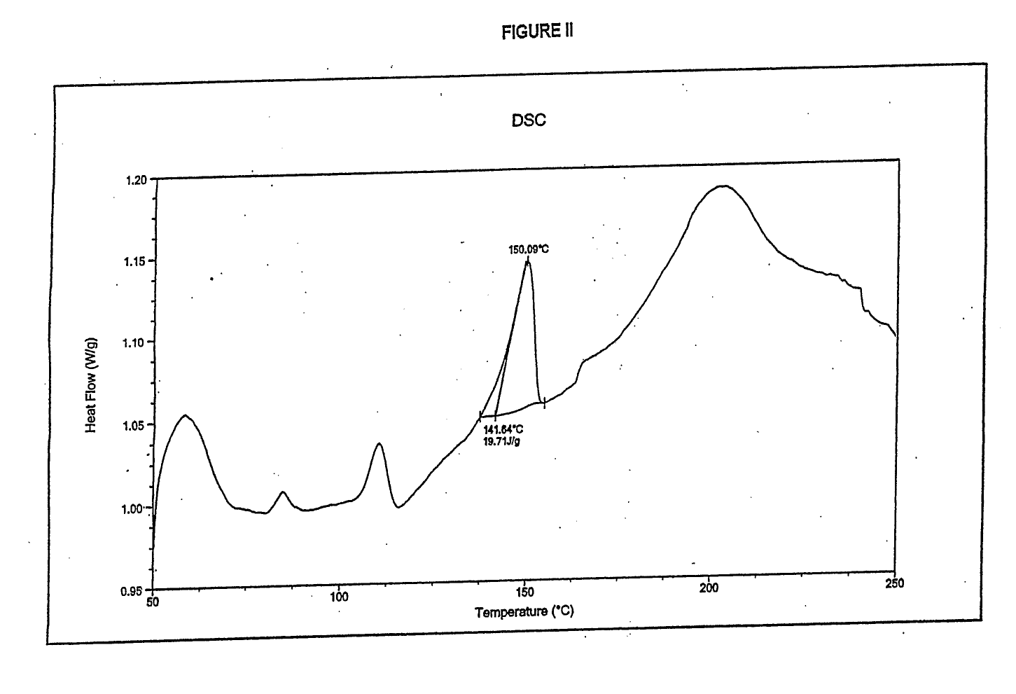

Crystalline Form S of Carvedilol dihydrogen phosphate of the present application is characterized by Differential Scanning Calorimetry (DSC) analysis thermogram pattern as shown substantially in FIG. 2, which shows endotherm at about 145 to about 153° C.

The crystalline form S of Carvedilol dihydrogen phosphate characterized further by an FT-IR spectrum that comprises at least one absorption band selected from the group consisting of 521.7, 727.1, 751.3, 786.9, 1024.2, 1051.2, 1100.4, 1125.5, 1219.0, 1253.7, 1332.8, 1454.3, 1505.4, 1605.7 and 1628.9±0.2 cm⁻¹.

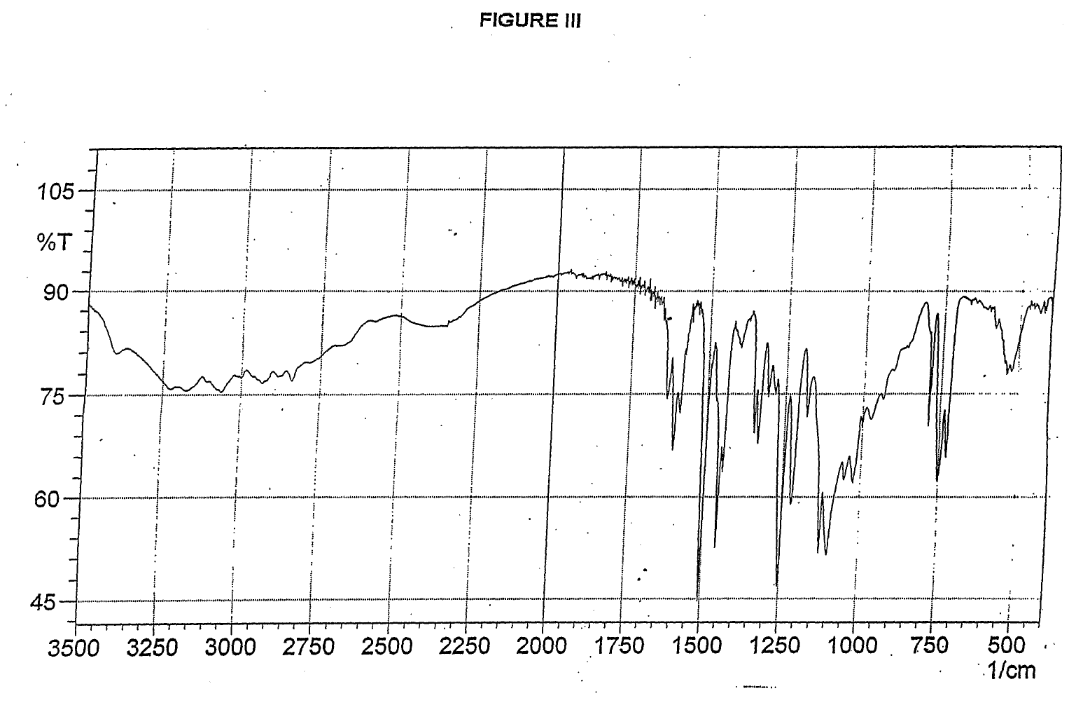

Crystalline Form S of Carvedilol dihydrogen phosphate of the present application is further characterized by an Infrared spectrum as shown substantially in FIG. 3.

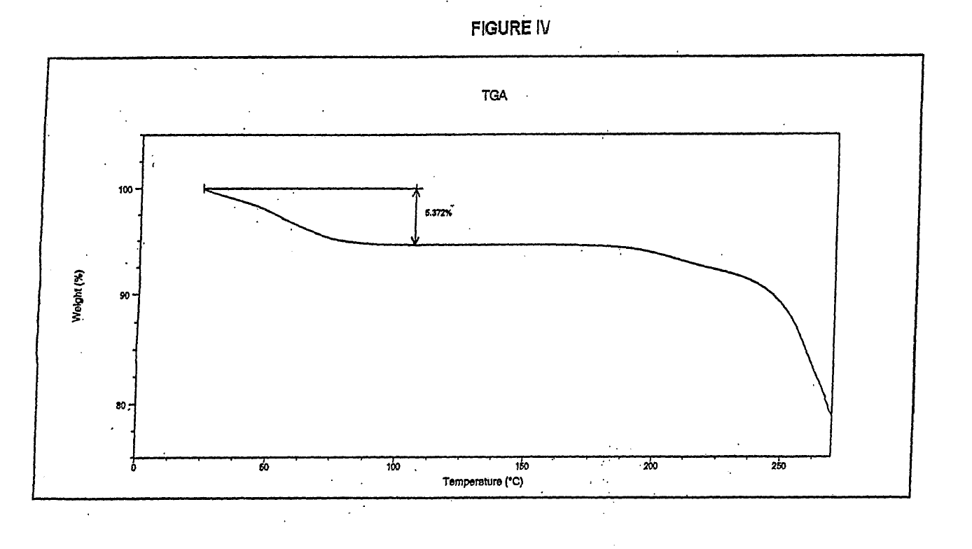

In another embodiment, crystalline Form S of Carvedilol dihydrogen phosphate obtained by the process of the present application is characterized by TGA curve substantially as illustrated in FIG. 4 corresponding to a weight loss of about 5.5% w/w.

In another embodiment, crystalline Form S of Carvedilol dihydrogen phosphate obtained by the process of the present application is characterized by moisture content up to about 5% by KF.

In second aspect the present application provides a process for the preparation of crystalline form of Carvedilol dihydrogen phosphate (Form S) comprising the steps of:

a) providing a solution of Carvedilol dihydrogen phosphate in a solvent comprising an alcohol;

b) causing the solution to precipitate; and

c) recovering the precipitated solid form.

In one embodiment, the step of providing a solution includes dissolving Carvedilol dihydrogen phosphate in a solvent. The dissolution may be carried out at a temperature suitable for complete dissolution of the components. The starting Carvedilol dihydrogen phosphate may be of any form such as crystalline, amorphous or mixture of crystalline and amorphous forms.

In another embodiment, the providing step includes dissolving free base of Carvedilol or a salt in a solvent, treating the free base or the salt solution with phosphoric acid to obtain Carvedilol dihydrogen phosphate solution.

In yet another embodiment, the providing step includes obtaining a reaction mixture in which Carvedilol free base is formed as product in a solvent and treating the free base solution with phosphoric acid to obtain Carvedilol dihydrogen phosphate solution.

The preferred solvents useful for providing solution include C1-C5 alcohols, and mixtures thereof. The particular solvents suitable for the providing step include methanol, ethanol, n-propanol, isopropanol, n-butanol, isobutanol, t-butanol and mixtures thereof. Methanol is most preferred.

The temperatures for providing solutions may range from about 20° C. to about 100° C. depending on the solvent used. Any other temperature is also acceptable as long as a clear solution of Carvedilol dihydrogen phosphate is obtained without affecting its quality. Preferably, dissolution is carried out at about 30° C. to about 70° C., preferably at about 40° C. to about 60° C.

The quantity of solvent used for providing solution depends on the solvent and the dissolution temperature opted for the process. The concentration of Carvedilol dihydrogen phosphate in the solution may generally range from about 0.1 to about 10 g/ml of the solvent.

The solution of Carvedilol dihydrogen phosphate is optionally treated with activated charcoal for about 10 to 30 minutes. The charcoal along with the undissolved particles may be removed suitably by filtration, centrifugation, decantation, and other techniques. Depending upon the equipment used, concentration and temperature of the solution, the filtration apparatus may need to be preheated to avoid premature crystallization.

Step b) involves causing precipitation from the solution.

Suitably the reaction solution is cooled to a lower temperature than the dissolution temperature to cause precipitation. In another variant, the solution may be concentrated to such an extent where precipitation is occurred or the solution may be concentrated followed by cooling to cause precipitation.

Step c) involves isolation of the precipitated solid form.

The precipitated solid may be isolated by any method including decantation, filtration by gravity or by suction, centrifugation, and the like. Other techniques for separating the solids from the reaction mixtures are also within the scope of this invention

The process may include further drying of the product obtained with or without vacuum and in presence or absence of inert atmosphere.

Drying may be suitably carried out in a tray dryer, rotavapour, air oven, fluidized bed drier, spin flash dryer, flash dryer and the like with or without vacuum. The drying may be carried out at temperatures of about 35° C. to about 100° C. The drying may be carried out for any time periods necessary for obtaining a desired quality, such as from about 5 minutes to several hours.

All XRPD data reported herein were obtained using a Bruker AXS D8 Advance Powder X-ray Diffractometer.

All TGA curves obtained from the present invention were carried out in a TGAQ500 of TA instruments (Lukens Drive, Del., USA).

Differential scanning calorimetric analysis was carried out on TAQ1000. The thermogram was recorded from 40° C. to 150° C. under the nitrogen flow of 50 mL/min at a heating rate of 5° C./min.

In third aspect there is provided a process for the preparation of Carvedilol dihydrogen phosphate comprises of:

a) reacting 4-(2,3-epoxy propoxy)carbazole with 2-(2-methoxy phenoxy)ethylamine to obtain a solution comprising Carvedilol;

b) reacting the Carvedilol solution obtained in step a) with phosphoric acid.

Suitable solvents useful for reaction of step a) includes ester solvents such as ethyl acetate, propyl acetate; ketone solvents such as acetone, methylethyl ketone, methyl isobutyl ketone; water or mixtures thereof in various proportions.

In one embodiment the reaction is carried out without using any external solvent.

Reaction of step a) is carried out at a temperature ranging from about 25° C. to about reflux temperature of the solvent used, preferably reflux temperature of the solvent used. Suitably reaction is maintained till completion of the reaction such as for about 10 to 30 hours, preferably for about 25 hours.

The molar ratio of 2-(2-methoxy phenoxy)ethylamine with respect to 4-(2,3-epoxy propoxy)carbazole ranges from about 1:0.5 to 1:5, preferably 1:2.5.

The reaction of step a) of reacting 4-(2,3-epoxy propoxy)carbazole with 2-(2-methoxy phenoxy)ethylamine is carried out in the absence of base, and also it can be carried out in the presence of a base. Suitable base that can be used includes sodium carbonate, potassium carbonate.

After completion of the reaction, the reaction mixture may be used directly in the next processing step or the solvent may be removed from the reaction mixture to obtain a residue.

The solvent removal may be carried out using suitable techniques such as distillation, evaporation with or without vacuum.

The residue is dissolved in a solvent preferably the same solvent used for the reaction and proceeded to next step

Step b) involves reacting the Carvedilol solution obtained in step a) with phosphoric acid.

Suitably phosphoric acid may be added directly or in the form of a solution either in water or a suitable organic solvent or a mixture thereof. Phosphoric acid may be added to the reaction mixture at once or slowly for a period of time or in lots with intervals.

The molar ratio of phosphoric acid with respect to 4-(2,3-epoxy propoxy)carbazole ranges from about 1:0.5 to 1:5, preferably 1:2.5.

Reaction of step b) is carried out at a temperature ranging from about 25° C. to about reflux temperature of the solvent used, preferably at ambient temperature

The reaction solution may be cooled to a lower temperature than the solution temperature to cause precipitation. In another variant, the solution may be concentrated to such an extent where precipitation is occurred or the solution may be concentrated followed by cooling to cause precipitation.

The Reaction mixture is maintained till complete precipitation of the product such as for about 30 hours, preferably for about 25 hours.

The precipitated solid may isolated by any method including decantation, filtration by gravity or by suction, centrifugation, and the like. Other techniques for separating the solids from the reaction mixtures are also within the scope of this invention

The process may include further drying of the product obtained with or without vacuum and in presence or absence of inert atmosphere.

In fourth aspect the present application provides pharmaceutical composition comprising crystalline form of Carvedilol dihydrogen phosphate (Form S) and pharmaceutically acceptable carrier.

Crystalline Form S of Carvedilol dihydrogen phosphate of the present application is sufficiently stable and suitable for preparation of pharmaceutical compositions.

In yet another aspect, the Carvedilol dihydrogen phosphate crystalline Form S of the present application may be formulated as solid compositions for oral administration in the form of capsules, tablets, pills, powders or granules. In these compositions. the active product is mixed with one or more pharmaceutically acceptable excipients. The drug substance can be formulated as liquid compositions for oral administration including solutions, suspensions, syrups, elixirs and emulsions, containing solvents or vehicles such as water, sorbitol, glycerine, propylene glycol or liquid paraffin.

The compositions for parenteral administration can be suspensions, emulsions or aqueous or non-aqueous sterile solutions. As a solvent or vehicle, propylene glycol, polyethylene glycol, vegetable oils, especially olive oil, and injectable organic esters, e.g. ethyl oleate, may be employed. These compositions can contain adjuvants, especially wetting, emulsifying and dispersing agents. The sterilization may be carried out in several ways, e.g. using a bacteriological filter, by incorporating sterilizing agents in the composition, by irradiation or by heating. They may be prepared in the form of sterile compositions, which can be dissolved at the time of use in sterile water or any other sterile injectable medium.

Pharmaceutically acceptable excipients used in the compositions comprising Carvedilol dihydrogen phosphate crystalline Form S of the present application include, but are not limited to diluents such as starch, pregelatinized starch, lactose, powdered cellulose, microcrystalline cellulose, dicalcium phosphate, tricalcium phosphate, mannitol, sorbitol, sugar and the like; binders such as acacia, guar gum, tragacanth, gelatin, pre-gelatinized starch and the like; disintegrants such as starch, sodium starch glycolate, pregelatinized starch, Croscarmellose sodium, colloidal silicon dioxide and the like; lubricants such as stearic acid, magnesium stearate, zinc stearate and the like; glidants such as colloidal silicon dioxide and the like; solubility or wetting enhancers such as anionic or cationic or neutral surfactants, waxes and the like. Other pharmaceutically acceptable excipients that are of use include but not limited to film formers, plasticizers, colorants, flavoring agents, sweeteners, viscosity enhancers, preservatives, antioxidants and the like.

Certain specific aspects and embodiments of the present application will be explained in more detail with reference to the following examples, which are provided by way of illustration only and should not be construed as limiting the scope of the invention in any manner.

### EXAMPLES

### Example 1

### Preparation of Carvedilol Dihydrogen Phosphate

In a dry reaction flask, 4-(2,3-epoxy propoxy)carbazole (50 gm. 0.21 moles), 2-(2-methoxy phenoxy)ethyl amine (75.5 gm. 0.45 moles) and 500 ml of ethyl acetate were charged and heated to reflux for about 24 hours. After completion of the reaction, solvent was distilled off from the reaction mixture to obtain 125 gm of the residue. Ethyl acetate (312 ml) and water (312 ml) were added to the residue and stirred for about 15 minutes. Reaction mixture pH was adjusted to about 3 with 40 ml (0.68 moles) of phosphoric acid at room temperature. Reaction mixture was stirred for about 11 hours and filtered the solid to obtain carvedilol phosphate. The wet solid thus obtained was slurred in 325 ml of acetone at 26° C. for about 30 minutes. The solid was filtered and dried to obtain the title compound.

Yield: 50 gm

### Example 2

### Preparation of Carvedilol Phosphate Crystalline Form S

Carvedilol phosphate (30 gm) and methanol (300 ml) were charged into a reaction flask and heated to 60-65° C. to obtain clear solution. The reaction solution was treated with activated carbon and filtered through Hyflow bed. About 165 ml of the filtrate was charged in to a clean flask and cooled to about 15° C. The reaction mixture was stirred for about 3 hours. The solid was filtered and washed with methanol. The wet solid was dried to obtain the title compound.

Yield: 8.5 gm

The obtained product obtained was analyzed by XRPD, DSC IR and TGA and the results are as provided in FIGS. 1, 2 3 and 4 respectively.

1. A crystalline form of Carvedilol dihydrogen phosphate (Form S) characterized by having an X-ray powder diffraction pattern comprising peak intensities expressed in degrees 2θ that are selected from 9.2±0.2, 13.0±0.2, 14.9±0.2, 19.7±0.2 and 28.1±0.2.

2. The crystalline form of Carvedilol dihydrogen phosphate according to claim 1, further having at least one additional X-ray powder diffraction peak selected from the group consisting of 6.6±0.2, 8.1±0.2 and, 22.2±0.2 degrees 2θ.

3. The crystalline form of Carvedilol dihydrogen phosphate according to claim 1, characterized by a single melting endotherm peak between about 145° C. and 153° C. as measured by differential scanning calorimetry.

4. The crystalline form of Carvedilol dihydrogen phosphate according to claim 1, further having an FT-IR spectrum that comprises at least one absorption band selected from the group consisting of 521.7, 727.1, 751.3, 786.9, 1024.2, 1051.2, 1100.4, 1125.5, 1219.0, 1253.7, 1332.8, 1454.3, 1505.4, 1605.7 and 1628.9±0.2 cm⁻¹.

5. The crystalline form of Carvedilol dihydrogen phosphate according to claim 1, having an X-ray powder diffraction pattern comprising diffraction peaks at 9.2±0.2, 13.0±0.2, 14.9±0.2, 19.7±0.2, 28.1±0.2, and a melting point in a range of about 145° C. to about 153° C.

6. The crystalline form of Carvedilol dihydrogen phosphate according to claim 1, characterized by X-ray powder diffraction pattern substantially as depicted in FIG. 1.

7. The crystalline form of Carvedilol dihydrogen phosphate according to claim 1, characterized by differential scanning calorimetry pattern substantially as depicted in FIG. 2.

8. A process for the preparation of crystalline form of Carvedilol dihydrogen phosphate (Form S) comprising the steps of:
a) providing a solution of Carvedilol dihydrogen phosphate in a solvent comprising an alcohol;
b) causing the solution to precipitate; and
c) recovering the precipitated solid form.

9. The process of claim 8, wherein said providing step comprises dissolving Carvedilol dihydrogen phosphate in said solvent.

10. The process of claim 8, wherein said alcohol solvent is C1-C5 alcohol.

11. The process of claim 10, wherein said alcohol solvent is selected from the group consisting of methanol, ethanol, n-propanol, isopropanol, n-butanol, isobutanol, t-butanol and mixtures thereof.

12. The process of claim 11, wherein said alcohol solvent is methanol.

13. A process for the preparation of Carvedilol dihydrogen phosphate comprises of:
a) reacting 4-(2,3-epoxy propoxy)carbazole with 2-(2-methoxy phenoxy)ethylamine to obtain a solution comprising Carvedilol;
b) reacting the Carvedilol solution obtained in step a) with phosphoric acid.

14. The process of claim 13, wherein the reaction of step a) is carried out in solvent is selected from ester solvent, ketone solvent and water or mixtures thereof.

15. The process of claim 14, wherein the reaction of step a) is carried out in solvent is selected from the group consisting of ethyl acetate, propyl acetate, acetone, methylethyl ketone, methyl isobutyl ketone and water or mixtures thereof.

16. The process of claim 13, wherein the reaction of step a) is carried out without using any external solvent.

17. A pharmaceutical composition comprising crystalline form S of the present application and a pharmaceutically acceptable carrier.
